(19) United States
(12) Patent Application Publication
(10) Pub. No.: US 2003/0216654 A1
(43) Pub. Date: Nov. 20, 2003

(54) Bayesian discriminator for rapidly detecting arrhythmias

(76) Inventors: Weichao Xu, Shenzhen (CN); Hung-Fat Tse, Midlevel (HK); Francis Hy Chan, Pokfulam (HK); Peter Chin Wan Fung, Pokfulam (HK); Chu-pak Lau, Midlevel (HK)

Correspondence Address: PENNIE AND EDMONDS, 1155 AVENUE OF THE AMERICAS, NEW YORK, NY, 100362711

(21) Appl. No.: 10/141,104

(22) Filed: May 7, 2002

(51) Int. Cl. A61B 5/04

(52) U.S. Cl. 600/509

(57) ABSTRACT

A method for accurate and rapid automated detection of atrial fibrillation (AF), sinus rhythm (SF), and atrial flutter (AFL) is disclosed, which allows distinguishing of these cardiac signals with lowered risk of errors by implanted pacemakers and like devices. The method includes training episodes of intra-cardiac signals (called the closed data set CDS) to evaluate five feature parameters with a discriminator classifying the signal into AF, AFL or sinus rhythm (SR). Comparison with the independent decisions of experienced physicians for each episode reveals specificity, accuracy and sensitivity of greater than 97%. Each episode is a window of intracardiac signal of interval 1-2 seconds with the discriminator providing results in less than 0.25 s. In another aspect, the method is resistant to the presence of noise in the data. In yet another aspect, more feature parameters may be used in alternative implementations including for detecting signals other than AF, AFL & SR.

### 1. FIELD OF THE INVENTION

The invention is directed to the generation and analysis of data with a multiple-index Bayesian discriminator. More specifically, the invention is directed to methods, systems, and devices for detecting and treating arrhythmias and heart diseases.

### 2. BACKGROUND OF THE INVENTION

2.1. Arrhythmias

Arrhythmias are caused by a disruption of the normal electrical conduction system of the heart, causing abnormal heart rhythms. Normally, the four chambers of the heart (2 atria and 2 ventricles) contract in a very specific, coordinated manner. The signal to contract is an electrical impulse that begins in the “sinoatrial node” (the SA node), which is the body's natural pacemaker. The signal then travels through the two atria and stimulates them to contract. The signal passes through the “atrioventricular note” node (the AV node), and finally travels through the ventricles and stimulates them to contract. Problems can occur anywhere along the electrical conduction system, causing various arrhythmias. There can be a problem in the heart muscle itself, causing it to respond differently to the signal, or causing the ventricles to contract out of step with the normal conduction system. Other causes of arrhythmias include abnormal rhythmicity of the body's natural pacemaker, a shift of the pacemaker from SA node to other parts, blocks at different transmission points, abnormal pathways of impulse conduction, and spontaneous general of abnormal impulses due to ischemia (low flow to coronary arteries), hypoxia (low oxygen), ANS imbalance, lactic acidosis, electrolyte abnormality, drug toxicity, and hemodynamic abnormalities.

Atrial fibrillation (AF) is the most common form of supraventricular arrhythmia and is associated with a considerable risk of morbidity and mortality. (Benjamin E J, et al., 1998 Circulation 98:946-952; Ryder K M, et al., 1999 Am J Cardiol. 84:1311R-138R; Chugh S S, et al. 2001 J Am Coll Cardiol. 37:371-377). As many as 2 million Americans are living with atrial fibrillation according to the American Heart Association. Theoretical analyses and high-density mapping studies have suggested that the most common mechanism of AF is the presence of multiple wave fronts or “wavelets” circulating irregularly throughout the atrial tissue. (Moe G K, et al., 1964 Am Heart J. 67:2961-2967; Allessie M A, et al., “Experimental Evaluation of Moe s Multiple Wavelet Hypothesis of Atrial Fibrillation” in Zipes E P, Jalife J, eds. Cardiac Electrophysiology and Arrhyhtmias. Orlando, Fla: Grune & Stratton, Inc., 1985; pp 265-275; Konings KTS, et al., 1994 Circulation 89:1665-1680).

Various studies have employed time domain, frequency domain, or time-frequency analysis to differentiate fibrillatory from non-fibrillatory rhythms. However, most of these, essentially single-index methods, techniques suffer from limitations such as rather long process time, lack of robustness to noise or far field events, poor performance in discriminating atrial flutter (AFL) from sinus rhythm (SR), and relatively low sensitivity and specificity.

These also limit improvements in pacemakers and other devices. For instance, in dual chamber pacemakers, accurate AF detection is critically important to avoid rapid ventricular pacing by activating automatic mode switching. In an implantable cardioverter defibrillator, accurate recognition of AF can avoid false discharges. Furthermore, the recent development of automatic implantable atrial defibrillators has created a critical need for speedy and reliable discrimination of AF from other types of intra-atrial electrograms. (Lau C P, et al., 1997 Pacing Clin Electrophysiol. 20:220-5; Wellens H I, et al., 1998 Circulation 98:1651-1656; Friedman P A, et al., 2001 Circulation. 104:1023-1028).

Proposed techniques for detecting AF can be conveniently divided into about four categories such as (1) methods based on time domain features (See Botteron G W, et al., 1996 Circulation 93:513-518; Botteron G W, et al., 1995 IEEE Trans. BME 42:579-586; Tse H F, et al., 1999 Circulation 99:1446-1451; Sih H J, et al., 1999 IEEE Trans. BME 46:440-450; Swerdlow C D, et al., 2000 Circulation 101:878-885; Thakor N V, et al., 1990 IEEE Trans. BME 37:837-843; Chen S W, et al., 1995 J Electrocardiol. S28:162; Chen S W, et al., 1996 IEEE Trans. BME 43:1120-1125); (2) methods employing frequency domain properties (See Ropella K M, et al., 1989 Circulation 80:112-119; Bollmann A, et al., 1998 Am J Cardiol 81:1439-1445; Chen S W, et al., 1996 ICASSP 963:1775-1778; Chen S W, 2000 IEEE Trans. BME 47:1317-1327); (3) techniques making use of time and frequency analysis (See Slocum J, et al. Computer discrimination of atrial fibrillation and regular atrial rhythms from intra-atrial electrograms. Pacing Clin Electrophysiol. 1988; 11:610-621; Lovett E G, et al., 1997 Ann BME 25:975-984); and (4) miscellaneous (See Zhang X S, et al., 1999 IEEE Trans. BME 46:548-555).

Botteron and Smith developed an algorithm based on the crosscorrelation of two pre-processed bipolar intra-atrial signals of which an active space constant was extracted (1996 Circulation 93:513-518; 1995 IEEE Trans. BME 42:579-586). Tse et al. depicted a two-phase AF detection method that directly processed the time domain signals (1999 Circulation 99:1446-1451). More recently, Sih et al. proposed an approach employing the mean square error in the linear prediction between two unipolar epicardial electrograms (1999 IEEE Trans. BME 46:440-450). Swerdlow et al. used a technique that combined the median cycle length and an atrial tachyarrhythmias evidence counter that used the number of sensed atrial electrograms in consecutive RR intervals (2000 Circulation 101:878-885). Chen et al. proposed a modified sequential algorithm based technique (1995 J Electrocardiol. S28:162; 1996 IEEE Trans. BME 43:1120-1125). Instead of measuring the rate, they employed blanking variability to measure the temporal irregularity with improved detection accuracy.

In addition to the time domain measures mentioned above, there are methods rooted in spectral analysis including coherence spectrum method and frequency analysis using the surface electrocardiogram. (See Ropella K M, et al., 1989 Circulation 80:112-119; Bollmann A, et al., 1998 Am J Cardiol 81:1439-1445). Chen et al. disclosed a two-stage arrhythmia discrimination method using a damped exponential modeling algorithm which gives higher frequency resolution than simple Fast Fourier transform methods (1996 ICASSP 963:1775-1778; 2000 IEEE Trans. BME 47:1317-1327). Similarly, Slocum et al. designed an algorithm that took into account both the morphological information (atrial rate and amplitude probability function) and frequency domain features (power spectrum analysis) (1988 Pacing Clin Electrophysiol. 11:610-621). In addition, Lovett and Ropella disclosed analysis of atrial rhythms via a time-frequency distribution of coherence (1997 Ann BME 25:975-984). From the viewpoint of dynamical systems, Zhang et al. proposed a complexity-based approach for discrimination of ventricular tachycardias and fibrillation (1999 IEEE Trans. BME 46:548-555), a method having a few advantages over the conventional detection techniques (Chen S W, 2000 IEEE Trans. BME 47:1317-1327). However, these methods need rather long episode (>5 s) to get satisfactory performance.

While techniques using single-index calculation are useful in the detection of arrhythmias, there is a continued need to find more accurate and rapid detection modalities and approaches to diagnose arrhythmias.

2.2. Statistical Analysis

2.2.1. Bayes Decision Rule

A Bayesian theorem describes the relationship that exists between simple and conditional probabilities. The Bayes decision theory assumes that the decision problem (whether an observed episode belongs to one class or another) is posed in probabilistic terms, and that all of the relevant probabilities are known. For instance, P(wᵢ) is denoted to be the prior probability that a certain episode should belong to wᵢ, i.e., P(wᵢ) is the probability that an episode is of class i even before it is observed. The symbol p(\vec{v}|wᵢ) denotes the class conditional probability of observing feature vector \vec{v} given the fact \vec{v} is of class wᵢ is known. In other words, p(\vec{v}|wᵢ) is a probability density function of non-negative value and can be estimated by the training data set. P(wᵢ|\vec{v}) is called the posterior probability which can be calculated by p(\vec{v}|wᵢ) and P(wᵢ) according to the Bayes' rule. P(wᵢ|\vec{v}) is the probability (between 0 and 1) that an object is of class wᵢ given it is observed as \vec{v}. If the cost of a correct decision is 0, and the cost of a wrong decision is 1, then, the Bayes Decision Rule can be applied as: Decide wᵢP(wᵢ|\vec{v})>P(wⱼ|\vec{v}) for all j≠i.

2.2.2. Sensitivity, Specificity, and Accuracy

Sensitivity and specificity together describe the accuracy of a test. When a large number of positive and negative samples are tested, sensitivity determines the percentage of false-negative results, and specificity determines the percentage of false-positive results. For example, a specificity of 99% means that 1% of those without AF will test false-positive for exhibiting AF. A sensitivity of 99%, on the other hand, means that 1% of those with AF will test false-negative, i.e., as not exhibiting AF.

### 3. SUMMARY OF THE INVENTION

Atrial fibrillation (AF) is the most common arrhythmia (abnormal heart beat) with a considerable risk of stroke and mortality. Atrial flutter (AFL) is another type of abnormal heart beat that also occur frequently in those patients with AF. Accurate and rapid detection of these rhythms is critically important to avoid rapid ventricular pacing by activating automatic mode switching and false shock discharges from implantable device (pacemaker and defibrillator). The detection of these abnormal rhythms by implantable devices require the use of intra-atrial electrograns recorded from the atria. Since the treatments of AF & AFL are clinically completely different, it is of rather urgent need that an algorithm is written to distinguish these three types of heart signals by the device. To train up our system of detection, several hundred episodes of intra-cardiac signals (called the closed data set CDS) were recorded by a computer. Five feature parameters were evaluated for each episode or window, and a discriminator is obtained to decide which class of signals (AF, AFL or sinus rhythm (SR), normal heart beat) does this episode belong to according to the mathematical method specified below. Experienced physicians make also a decision for each episode independently. The two results are then compared. The performance of this algorithm as specified by the specificity, accuracy and sensitivity. After checking these three to be satisfactory (>97%), the statistical averages of the five feature parameters are calculated and the system is ready to use.

Our new algorithm allows decision to be made based on a window of intracardiac signal of interval 1-2 seconds only and the computer calculation time is shorter than 0.25 s. Any new set of episode is added to the CDS. Since the performance of the algorithm improves as the size of CDS is increased, this algorithm gets “smarter” as more cases are tested.

To check the performance, we have used several hundred episodes of rhythms called open data set (ODS, different from CDS) and have found that our methodology works well. We impose noise to the ODS and found that the algorithm has very good anti-noise property.

Note that we have used five feature parameters based on the physical interpretation specified later. This number five can be extended to higher number for better result, when we find other interpretations or when we treat signals other than AF, AFL & SR. Moreover, based on information from one or few windows (˜1 second) of signals, the calculation time has to be very short (preferably <1 s) so that the implantable device can use the information and make decision on the type of treatment on line. Our invention marks the basis of producing software to be attached to machines associated with intracardiac signal detection.

### 4. BRIEF DESCRIPTION OF FIGURES

FIGS. 1A-C illustrate the various feature extraction for episodes of SR (FIG. 1A), AF (FIG. 1B), and AFL (FIG. 1C) including (a) raw episode; (b) output after manipulations 1 to 3; (c) auto-correlation coefficients; and (d) rectified version read for feature extraction.

FIG. 2 shows a flowchart of the steps involved in the training and discrimination procedure. Block arrows indicate the training process and solid arrows indicate the detection procedure.

FIG. 3 shows the comparison of values of features for open and close data sets. White, black, and shadowed bars represent SR, AFL, and AF, respectively. Each of the five features are significantly different between AF, AFL, and SR for both open and close data sets. There are no significant differences in the values of each of the five features for AF, AFL, and SR between close and open data set.

FIG. 4 shows the performance (e.g., sensitivity, specificity, and accuracy) achieved according to the number of features used.

FIG. 5 shows the relationship between the performance (e.g., sensitivity, specificity, and accuracy) of the disclosed discriminator and the signal-to-noise ratio (SNR).

### 5. DETAILED DESCRIPTION OF THE INVENTION

The present invention generally relates to methods, systems, and devices for detecting and treating arrhythmias and heart diseases. Atrial tachyarrhythmias are detected in a subject using a multiple-index Bayesian discriminator. The method for detection comprises the steps of obtaining an open-test data set of bipolar intra-atrial signals from the subject of interest and using a computer or computers to analyze the open-test data set. Furthermore, the method for detection generates a result in accordance with a set of estimated conditional probabilities from a training data set based on the multiple-index Bayesian discriminator. The use of a computer, or a computing device system in practicing the method is illustrative and includes any computer executable processing device. Similarly, the method is suitable for detecting various conditions such as sinus rhythm, atrial flutter, atrial fibrillation, or any type of arrhythmias, heart diseases, or physiological conditions. In general, the open-test data set may comprise any type of electophysiological information (e.g., ECG, EEG, and EKG) obtained from the subject of interest although ECG data is employed in the preferred embodiment.

In another embodiment, the method for detection further comprises the steps of selecting a plurality of features of intra-atrial electrograms and a type of output, inputting a close-test data set of bipolar intra-atrial signals for training, and estimating the set of conditional probabilities for the plurality of features and the type of output in accordance with a multiple-index Bayesian discriminator from the close-test data set. Of course, the method described herewith is applicable to any type of electophysiological information (e.g., ECG, EEG, and EKG) obtained from the subject of interest.

In another embodiment, the method for detection further comprises the step of selecting additional features for estimating conditional probabilities. The plurality of features of intra-atrial electrograms may be selected from the non-exhaustive illustrative list comprising regularity, rate, energy distribution, percent time of quiet interval, and number of baseline reaching. For instance, the plurality of features may also be selected from those parameters disclosed in previous studies such as cross-correlation of two pre-processed biopolar intra-atrial signals (Botteron GW and Smith J M, 1995 IEEE Trans. BME 42:579-586; Botteron G W and Smith J M, 1996 Circulation 93:513-518), time (Tse H F, et al., 1999 Circulation 99:1446-1451; Thakor N V, et al., 1990 IEEE Trans. BME 37:837-843), mean square error in the linear prediction between two unipolar epicardial electrograms (Sih H J, et al., 1999 IEEE Trans. BME 46:440-450), median cycle length in conjunction with the number of sensed atrial electrograms in consecutive RR intervals (Swerdlow C D, et al., 2000 Circulation 101:878-885), temporal irregularity (Chen S W, et al., 1995 J Electrocardiol. S28:162; Chen S W, et al., 1996 IEEE Trans. BME 43:1120-1125), and frequency (Ropella K M, et al. 1989 Circulation 80:112-119; Bollmann A, et al. 1998 Am J Cardiol 81:1439-1445; Chen S W, et al., 1996 ICASSP 963:1775-1778; Chen S W, 2000 IEEE Trans. BME 47:1317-1327).

In another embodiment, the method for detection further comprises the step of modifying at least one estimated conditional probabilities from the set of estimated conditional probabilities. Preferably, the open-test data set and the results obtained from analysis of the open-test data set are incorporated into to the closed-test data set in an iterative manner. The set of estimated conditional probabilities is continuously modified as more data set is inputted. Thus, performance of the method can be continuously modified or improved, i.e., increasing the specificity, sensitivity, and accuracy of the result.

In another embodiment, the method for detection further comprises the step of differentiating between the types of arrhythmias or heart diseases in the subject of interest. To this end, a sufficient number of features of intra-atrial electrograms are used so the method for detection displays an overall sensitivity of at least 90%, preferably 95%, more preferably 98%, and most preferably 99%, an overall specificity of at least 90%, preferably 95%, more preferably 98%, and most preferably 99%, and an overall accuracy of at least 90%, preferably 95%, more preferably 98%, and most preferably 99%. An illustrative non-exhaustive list of arrhythmias detected by the disclosed method includes sinus rhythm, atrial flutter, atrial fibrillation, atrial tachyarrhythmias, tachycardia, bradycardia, supraventricular arrhythmias, premature atrial contractions (PACs), paroxysmal supraventricular tachycardia (PSVT), accessory pathway mediated tachycardias, atrial tachycardia, ventricular arrhythmias, premature ventricular contractions (PVCs), ventricular tachycardia, ventricular fibrillation, bradyarrhythmias, sinus node dysfunction, and heart block.

In another embodiment, the method for detection shows robust anti-noise performance in differentiating between atrial fibrillation (AF), atrial flutter (AFL), and sinus rhythm (SR). The overall sensitivity, specificity, and accuracy of a method for detection is similar at different signal-to-noise ratio (SNR) above 10 dB. The overall sensitivity of the method for detection is at least 90%, preferably 95%, more preferably 98%, and most preferably 99% when the SNR is greater than 10 dB. Similarly, the overall specificity of the method for detection is at least 90%, preferably 95%, more preferably 98%, and most preferably 99% and the overall accuracy of the method for detection is at least 60%, 65%, 70%, 75%, 80%, 85%, 90%, or 95% when the SNR is greater than 10 dB.

In another embodiment, the method further comprises the step of providing a treatment in response to detecting a particular condition. Such treatment options include, but are not limited to, medications, cardioversion, pacemakers, implantable cardioverter-defibrillators, surgery, or radiofrequency catheter ablation of the arrhythmia focus. In particular, an implanted device that can adjust its stimulation in response to rapidly detecting a particular arrythmia. Such rapid detection is enabled in less than five seconds, more preferably in less than 4 seconds, even more preferably less than 3 seconds and most preferably less than 2 seconds including at least one of 1.9 secs., 1.8 secs., 1.7 secs., 1.6 secs., 1.5 secs., 1.4 secs., 1.3 secs., 1.2 secs, 1.1 secs., 1.0 secs., 0.9 secs., 0.8 secs., 0.7 secs., 0.6 secs., 0.5 secs., 0.4 secs., 0.3 secs., 0.2 secs, and 0.1 secs.

In another embodiment, a device detects arrhythmias in a subject of interest. The device for detection comprises a module for collecting an open-test data set of bipolar intra-atrial signals from the subject of interest and a computer or a system of computer devices for analyzing the open-test data set. Furthermore, the device for detection comprises a screen or similar device that can display the results in accordance with a set of estimated conditional probabilities. The open-test data set can be collected in any tangible or intangible database or storage means. The module need not be a separate or discrete unit; it can be a program, a processor, a sub-component, etc. Further, the analysis could be carried out by any computer executable processing device and not just a computer. Similarly, the device could be used to detect sinus rhythm, atrial flutter, atrial fibrillation, or any type of arrhythmias, heart diseases, or physiological conditions.

In another embodiment, the device for detection further comprises a module, wherein the module selects a plurality of features of intra-atrial electrograms and a type of output, inputs a close-test data set of bipolar intra-atrial signals for training, and estimates the set of conditional probabilities for the plurality of features and the type of output in accordance with a multiple-index Bayesian discriminator from the close-test data set. The device for detection further comprises a third module, wherein the module selects additional features for estimating conditional probabilities. Possible features of intra-atrial electrograms for analysis include the features in the group consisting of regularity, rate, energy distribution, percent time of quiet interval, and number of baseline reaching, cross-correlation of two pre-processed biopolar intra-atrial signals, time, mean square error in the linear prediction between two unipolar epicardial electrograms, median cycle length in conjunction with the number of sensed atrial electrograms in consecutive RR intervals, temporal irregularity, and frequency.

A module may perform all or a sub-combination of steps, i.e., collecting data set, analyzing data set, providing an analysis, selecting a plurality of features, selecting a type of output, estimating a set of conditional probabilities, and displaying the intermittent and/or final results. Further, the analysis could be carried out by any computer executable processing device or devices. Furthermore, the module may include facility for modification of an estimated conditional probabilities from the set of estimated conditional probabilities. In order to so modify any conditional probability, preferably, the open-test data set and the results obtained from analysis of the open-test data set are added to the closed-test data set in an iterative manner. The set of estimated conditional probabilities is continuously updated as more data set is inputted. Thus, the performance of the method is continuously modified or improved, i.e., increasing the specificity, sensitivity, and accuracy of the result. Of course, more than one estimated conditional probabilities may be improved upon in like manner.

In another embodiment, a device for detection further comprises a module, wherein the module differentiates between the types of arrhythmias or heart diseases in the subject of interest. In a preferred embodiment, the module uses a sufficient number of features of intra-atrial electrograms so the device for detection displays an overall sensitivity of at least 90%, preferably 95%, more preferably 98%, and most preferably 99%, an overall specificity of at least 90%, preferably 95%, more preferably 98%, and most preferably 99%, and an overall accuracy of at least 90%, preferably 95%, more preferably 98%, and most preferably 99%. The different types of arrhythmias include, without limitation, sinus rhythm, atrial flutter, atrial fibrillation, atrial tachyarrhythmias, tachycardia, bradycardia, supraventricular arrhythmias, premature atrial contractions (PACs), paroxysmal supraventricular tachycardia (PSVT), accessory pathway mediated tachycardias, atrial tachycardia, ventricular arrhythmias, premature ventricular contractions (PVCs), ventricular tachycardia, ventricular fibrillation, bradyarrhythmias, sinus node dysfunction, and heart block.

In yet another embodiment, a device for detection further comprises a member that provides a modulating effect on heartbeats corresponding to the result. For instance, the member can deliver an electrical signal or input to the chest wall that synchronizes the heart and allows the normal rhythm to restart (as in a electrical cardioversion). Or, the member can send small electrical impulses to the heart muscle to maintain a suitable heart rate (like a pacemaker), deliver energy to the heart muscle to cause the heart to beat in a normal rhythm (like an implantable cardioverter-defibrillator), and even direct applying or delivering of high radio-frequency energy through a special catheter to small areas of tissues that cause abnormal heart rhythms (as in radiofrequency catheter ablation). Moreover, this description of the member is illustrative rather than limiting. For instance, different types and combinations of pacemakers and implantable cardioverter-defibrillators can be directly incorporated into the device. Additional technology for modulating (i.e., increases, decreases, stabilizes) heart rhythms can be incorporated into the device without limitation to respond to the detection of a particular arrhythmia. Such technology can include pharmaceutical, biological, chemical, physiological, electrical, anatomical, and molecular (i.e., antibodies, anti-antibodies, fusion proteins, polypeptides, fragments, homologues, derivatives, and analogues thereof) possibilities.

The subjects to which the methods, systems, and devices for detection and treatment of the present invention are applicable may be to any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human. Additional teachings are clarified with the aid of details in an example study below.

### 5.1. EXAMPLES

5.1.1. Data Acquisition

Bipolar intra-atrial electrograms at high anterolateral right atrium (with a 1 cm inter-electrode distance) from 20 patients in AF, AFL and SR were amplified and recorded (CardioLab 4.11, Pruka Engineering, Inc.) during electrophysiological procedures. The patients were presented to the electrophysiology laboratory for internal cardioversion of AF, electrophysiology study and/or radiofrequency ablation procedure for their underlying arrhythmias. Up to 220 seconds (mean: 190±20 seconds; range: 180 to 220 seconds) of simultaneous unfiltered (band pass 0.04-5000 hertz) recording from each patient were digitized at 1000 hertz. The data was then split into 1 (AF & AFL) or 2 seconds (SR) segments for analysis so that at least two atrial events were recorded during SR. In order to generate an unbiased data set, nearly the same numbers of episodes were randomly collected from each patient. Computer processing was performed using a Matlab 5.3 computer program (The Mathwork, Inc.).

The example study consisted of 20 patients (17 men and 3 women, mean age 55±16 years, ±SD). Their mean left ventricular ejection fraction was 56±10%, and their mean left atrial diameter was 4.6±1.7 cm as measured by echocardiography. Their clinical characteristics are summarized in TABLE 1.

TABLE 1
Patients Characteristics

| | | | | | Rhythm | |
|---|---|---|---|---|---|---|
| Patient | Age | Sex | Diagnosis | Medications | recorded | Procedure |
| 1 | 50 | M | HT, AF | CCB, Amiodarone | SR, ST, AF | Internal CV |
| 2 | 55 | M | Lone AF | Amiodarone | SR, AF | Internal CV |
| 3 | 68 | M | HT, AF | BB, Amiodarone | SR, AF | Internal CV |
| 4 | 55 | M | HT, AF | ACEI, Amiodarone | SR, AF | Internal CV |
| 5 | 60 | M | Lone AF | CCB | SR, ST, AF | Internal CV |
| 6 | 53 | M | HT, AF | ACEI, Amiodarone | SR, AF | Internal CV |
| 7 | 72 | F | Congestive heart | ACEI, Digoxin, | SR, AF | Internal CV |
| | | | failure, AF | Amiodarone | | |
| 8 | 48 | M | Pericarditis, AFL | Sotalol | SR, Typical | EP/RF |
| | | | | | AFL | |
| 9 | 53 | M | Coronary artery | BB, CCB | SR, ST, | EP/RF |
| | | | disease, AFL | | Typical AFL | |
| 10 | 70 | M | Lone AFL | None | SR, | EP/RF |
| | | | | | Atypical AFL | |
| 11 | 66 | M | HT, AF | CCB, Amiodarone | SR, AF | Internal CV |
| 12 | 67 | M | HT, AFL | CCB | SR, Typical | EP/RF |
| | | | | | AFL | |
| 13 | 64 | M | Lone AFL | CCB | SR, Atypical | EP/RF |
| | | | | | AFL | |
| 14 | 40 | M | Lone AF | Amiodarone | AR, AF | Internal CV |
| 15 | 66 | M | HT, AF | CCB, Amiodarone | SR, ST | Internal CV |
| 16 | 50 | M | AVNRT | None | SR, ST | EP/RF |
| 17 | 56 | F | WPW | None | SR, ST | EP/RF |
| 18 | 14 | F | WPW | None | SR, ST | EP/RF |
| 19 | 21 | M | WPW | None | SR, ST | EP/RF |
| 20 | 70 | M | AVNRT | None | SR, ST | EP/RF |

Abbreviations: AF, atrial fibrillation; AFL, atrial flutter; AVNRT, atrioventricular nodal reentry; BB, beta-blocker; CAD, coronary artery disease; CCB, calcium channel blocker; EP, electrophysiology study; HT, hypertension; RF, radiofrequency ablation; SR, sinus rhythm; ST, sinus tachycardia; WPW Wolff-Parkinson-White syndrome.

A total of 364 bipolar recording were collected from these patients. All rhythm episodes have been assessed blindly and classified into AF, AFL or SR by 2 experienced electrophysiologists. Of these recording, 156 episodes were AF, 88 episodes were AFL (mean atrial cycle length 320±40 ms, range 290-345 ms), and 120 episodes were SR, including 50 episodes of sinus tachycardia during isoprenaline infusion (mean sinus cycle length 535±30 ms, range 505-570 ms). Each patient contributed nearly the same number of episodes to the data set (18-22 episodes per patient). We randomly selected 219 (60%) and 145 (40%) rhythms as close-test data set and open-test data set, respectively.

5.1.2. Signal Manipulation

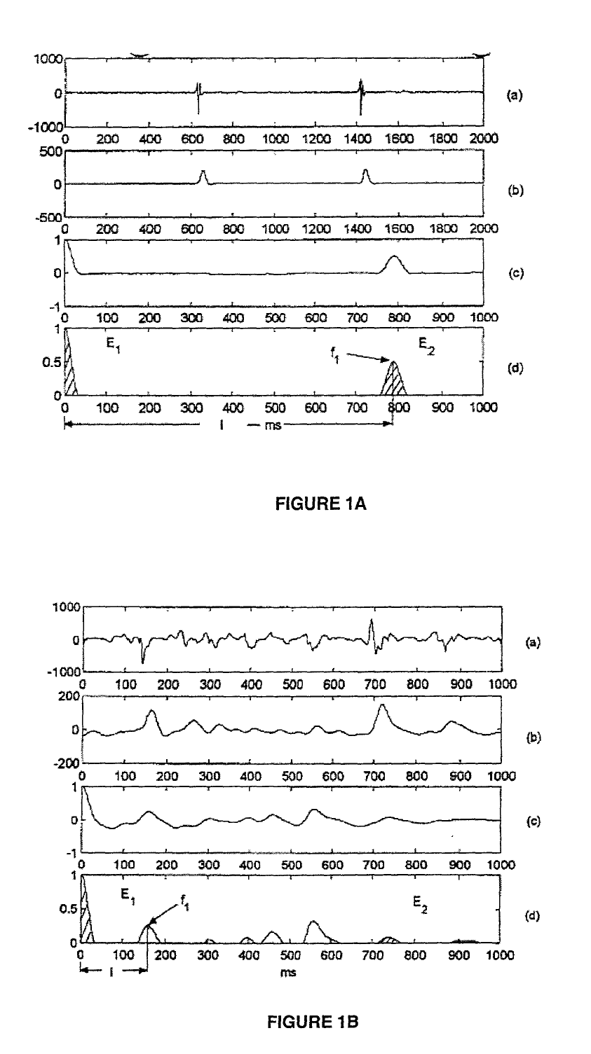

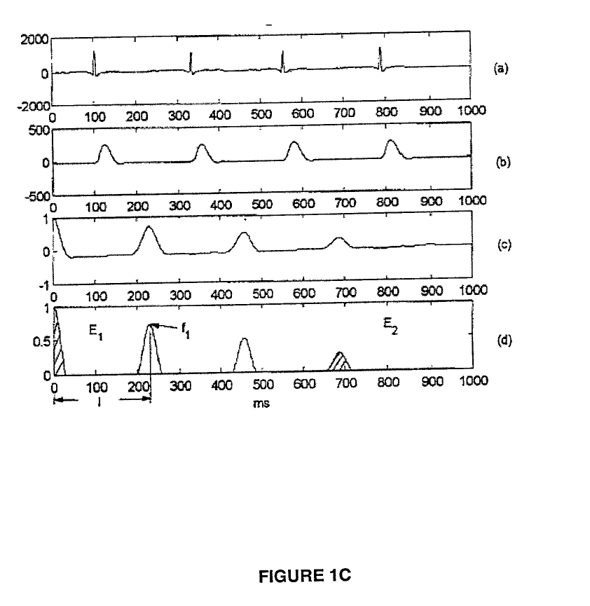

Before extracting the features of the signal, each rhythm episode was processed with the following manipulations: (1) third-order Butterworth bandpass filtering (40-250 Hz), (2) absolute valuing, (3) low pass filtering (0-20 Hz), (4) autocorrelation, and (5) rectification (FIG. 1). Steps 1 to 3 output a flattened signal proportional to the high frequency energy contained in the input episode. (Botteron G W and Smith J M, 1996 Circulation 93:513-518; Botteron G W and Smith T M, 1995 IEEE Trans. BME 42:579-586). The autocorrelation process avoids drastic fluctuation of the amplitude of atrial electrograms with time. (Oppendheim A V, Schafer R W. In: Discrete-Time Signal Processing, Chapter 11, Prentice-Hall International, Inc., 1989:742-756. Krauss T P, Shure L, Little J N. In: Signal Processing Toolbox User's Guide, Chapter 1, The Math Works Inc., 1994:61-63). Finally, the rectification process removes all the negative parts of the processed signal to facilitate the mathematical treatment during feature extraction.

5.1.3. Feature Extraction Procedure

Five relevant feature parameters were extracted from the final processed signal by a feature extraction procedure (FIG. 1). The first feature (f₁) is defined as the first peak, occurring at time (t), which is positively related to the regularity of the input. The second feature (f₂) is defined as f₂=t/1000, and is proportional to the input's atrial rate. The third feature (f₃) is defined as the percentage of energy contained in the two time bands (E₁+E₂/E), where E₁, is the energy within 0 to 100 ms, E₂ is the energy within 500 ms to 1000 ms, and E is the total energy within 0 to 1000 ms. The typical sinus rate is measured at 60-120 beats per minutes, i.e., the corresponding peak to peak interval is 500-1000 ms. In SR, the energy is mainly distributed in the aforementioned two time bands. Therefore, feature f₃, is helpful to distinguish SR signals from the other two classes of rhythm (AF or AFL) since the value of f₃ is very close to one for SR and smaller for AF or AFL. The fourth feature (f₄) measures the percent time interval corresponding to zero amplitude signal (percent quiet interval) and is calculated by the sum of time intervals with zero value over the total duration of rectified auto-correlation function. The fifth feature (f₅) measures the number of components that reaching the baseline in 1 second (baseline reaching). Both features f₄ and f₅ reflect the chaotic extent or randomness of the input signals and therefor, are supposed to be sensitive to fibrillatory rhythm (AF). The entire group of parameters f₁, f₂, f₃, f₄ and f₅ form a vector in five dimensions, which can only be determined if all the values of these 5 variables are known.

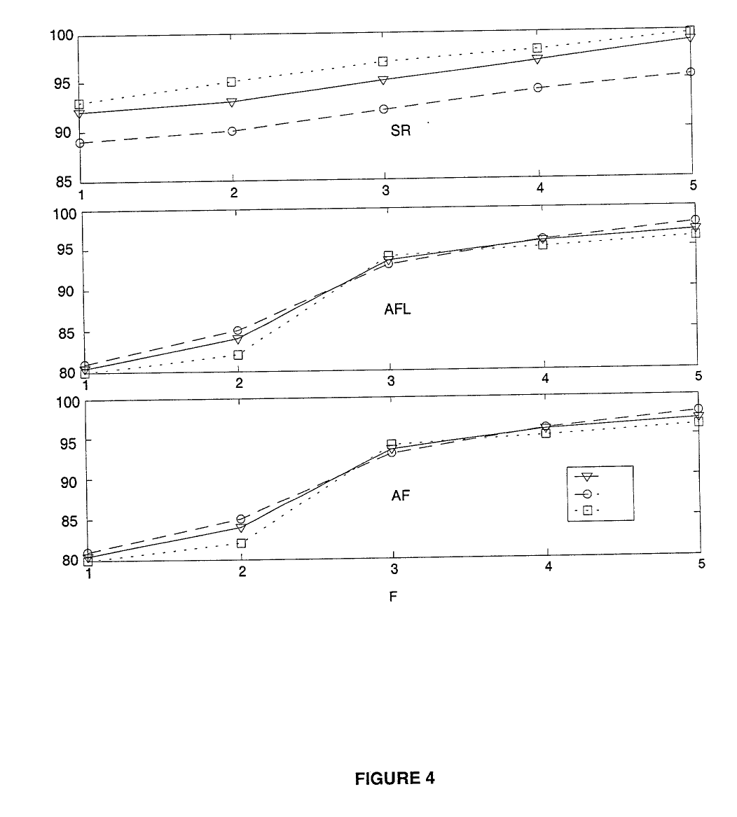

FIG. 4 shows respectively the sensitivity, specificity and accuracy of rhythm detection versus the increase of features. With the number of feature(s) used increase from 1 to 5, the performance increases significantly (p<0.01) from around 80% to above 95. This result also indicates the advantage of multi-feature detection over single-feature detection.

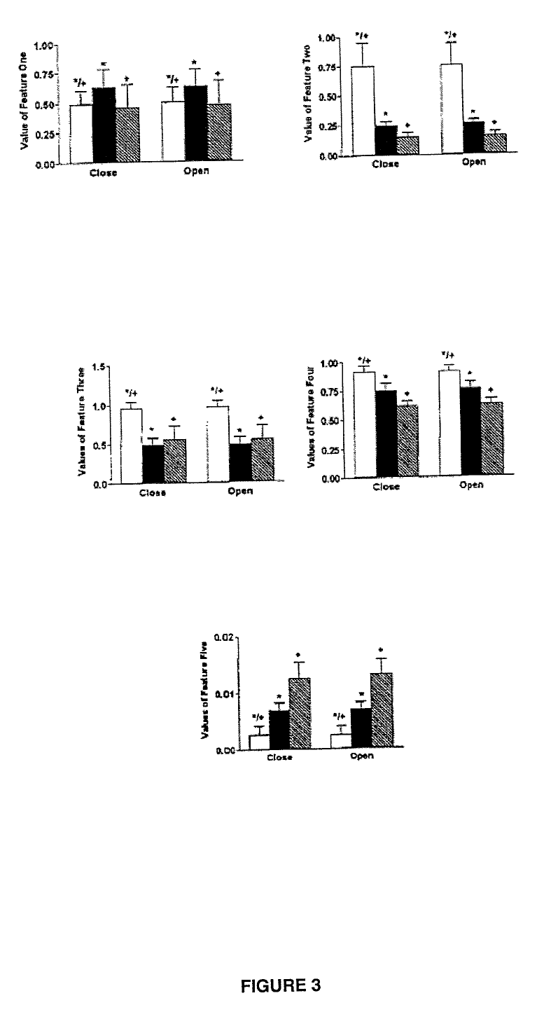

The results of 5 extracted features for the close and open data set are presented in FIG. 3. The values of each of 5 features were significantly different between AF, AFL and SR for both close and open data set. However, there are also significant overlaps between the values among the three types of rhythm for each feature. There were no significant differences in the values of each of 5 features for AF, AFL and SR between close and open data set, suggesting the two data sets were very similar.

5.1.4. Training Process

Sixty percent of the collected rhythm episodes were randomly selected as the closed-test training data set of the new discriminator. The values of f₁, f₂, f₃, f₄ and f₅, and the corresponding feature vector for the three classes of rhythm signals (SF, AF, and AFL) were obtained. The distribution of each of the five features has been found to follow approximately the normal distribution, therefore, the corresponding feature vectors of each class of rhythm also satisfy approximately a 5-dimensional normal distribution. Similar to the one-variate normal distribution, the multi-variate normal distribution is also determined by two parameters—mean and the so-called Covariant Matrix, both of which could be estimated via the feature vectors of the training data set (close-test data set). The mean and the Covariant Matrix are both necessary for the discrimination procedure depicted in the following section 6.1.5.

The objective of training process is to estimate the prior probability P(wᵢ) and the class distribution p(\vec{v}|wᵢ). These two items are necessary for calculating the posterior probability P(wᵢ|\vec{v}), which is critical for the discrimination procedure. In practice, P(wᵢ) can be approximated by nᵢ/Σⱼ₌₁³nⱼ, where nᵢ is the total episode number of the i^{th} class. P(wᵢ|\vec{v}) can be calculated by p(\vec{v}|wᵢ) P(wᵢ) according to Bayes' rule. Assume that p(\vec{v}|wᵢ) is normal, the following equation (1) is obtained:

$$p ( \vec{v} \vert {w}_{i} ) = \frac{1}{{( 2 π )}^{5 / 2} {\vert \sum \vert}^{1 / 2}} exp [ \frac{{\vec{v} - \vec{u} )}^{t} {\sum }^{- 1} ( \vec{v} - \vec{u} )}{2} ] , \qquad ( 1 )$$

where \vec{μ}=E[\vec{v}] is the mean of v, and Σ=E [(\vec{v}−\vec{μ})^{t}] is the covariant matrix generated by the vector (\vec{v}−\vec{μ}); t denotes transpose and −1 denotes inverse of a matrix.

5.1.5. Discrimination Procedure

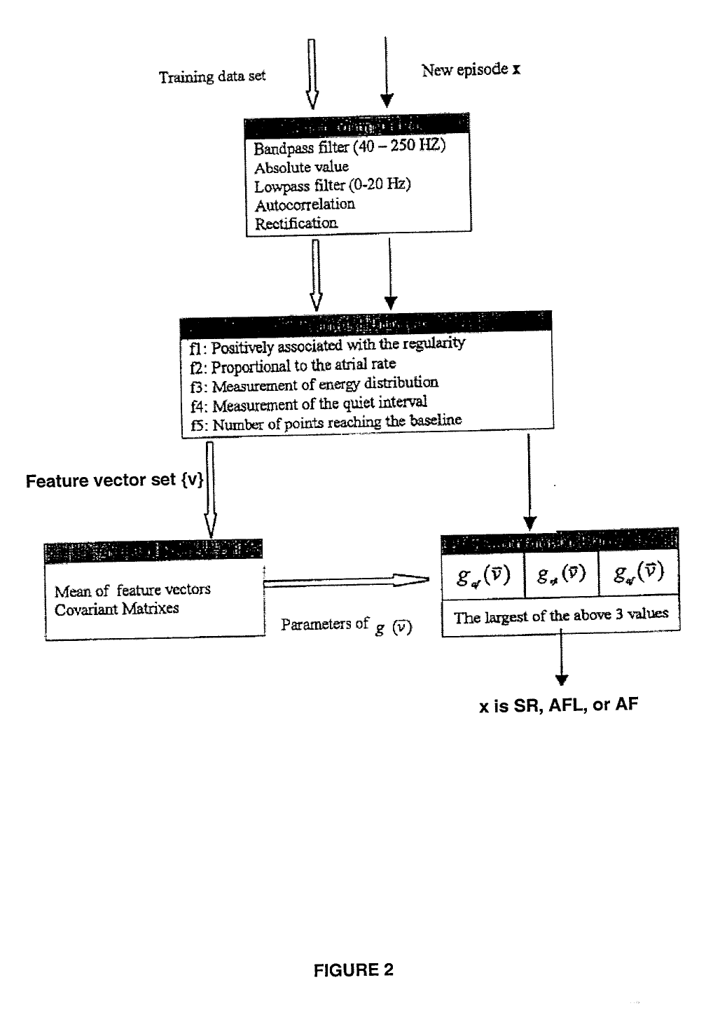

In order to optimize detection performance, a multi-variate Bayes decision theory is used. (See section 2.2.1). Using the Bayes Theorem, the posterior probability, which is the chance that a feature vector of any episode should belong to any of the three classes of rhythm, is calculated. Then, a so-called “discrimination function, g(\overline{v})” or a class of rhythm in general based on Bayes decision theory, is generated. For each rhythm episode, the values of the three discrimination functions g_{SR}(\overline{v}), g_{AF}(\overline{v}), g_{AFL}(\overline{v}), which correspond to the probabilities of the episode belonging to SR, AF, and AFL, respectively, are evaluated. The final decision for each rhythm episode is simply determined by which of absolute value of the above three is the largest (FIG. 2). The detailed mathematical treatment leading to the representation of the discrimination function is discussed below.

Theoretically, the detection process is to calculate the posterior probabilities P(wᵢ|\vec{v})=p(\vec{v}|wᵢ)P(wᵢ) of all 3 classes given one unknown episode. However, because normal distribution has exponential terms, which is time-consuming to calculate, for computation efficiency, the logarithm on both side of the above equation is taken:

gᵢ(\vec{v}=log P\vec{v}|wᵢ)+log P(wᵢ) (2)

Then, equation (1) is substituted into equation (2), obtaining a convenient form for the “discrimination function” gᵢ(\vec{v}):

gᵢ(\vec{v})=\vec{v}^{t}Wᵢ\vec{v}+wᵢ^{t}\vec{v}+wᵢₒ (3)

where Wᵢ=−½Σᵢ⁻¹ (4)

\vec{w}=Σᵢ⁻¹\vec{μ}_{I} (5)

wᵢₒ=−½\vec{μ}ᵢ^{t}Σᵢ⁻¹\vec{μ}ᵢ−½ log e|Σ_{I|+log}ₑ P(wᵢ) (6)

After calculating the three values of gᵢ \vec{v} (i=1,2,3), the i value corresponding to the maximum gᵢ is chosen according to the Bayes decision rule.

5.1.6. Anti-Noise Performance

Sometimes the intracardiac signals may be corrupted by noises introduced by external electromagnetic interference and myopotential sensing. It is important for the method to be robust when processing noisy episodes. As shown in this study, the SNR has significant effect on the performance of the disclosed discriminator. A decrease in SNR reduces the sensitivity for detection of regular rhythms, such as SR and AFL. This phenomenon is due to the “noisy nature” of AF signals. The additive noises increase the randomness of all three classes of signals, which makes a discriminator to judge all episodes as AF, hence favors AF class. As a result, the specificity for detection of AF also decreases as the SINK reduces. This new Bayesian Discriminator has satisfactory performance (over 95%) for detection of SR. AFL and AF when the SNR≧10 dB.

To test the anti-noise performance of the disclosed discriminator, Gaussian white noises were intentionally added with different signal-to-noise ratio (SNR) to each episode of the close test data set.

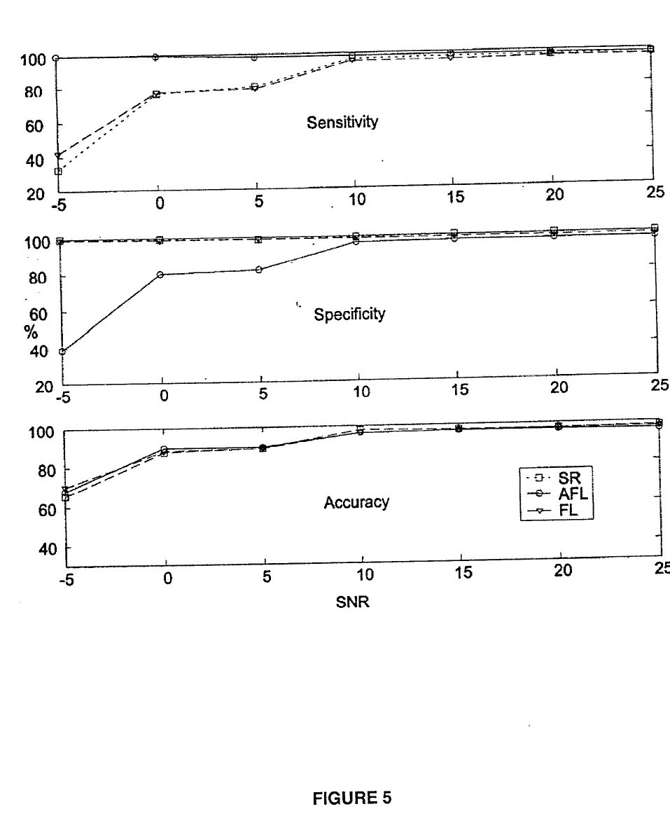

The effects caused by increasing the SNR on the performances of the new Bayesian Discriminator are presented in FIG. 5. With a decrease in SNR, the sensitivity for detection of more regular rhythms as SR and AFL decreased accordingly, while the sensitivity for AF detection remained at high levels. However, the specificity for AF detection decreased with the reduction of SNR, while the specificity for SR and AFL detection remained at high levels. As a result, the overall accuracy for detection of SR, AFL and AF are similar at different SNRs. When the SNR is greater than 10 dB, the disclosed discriminator has an accuracy of about 95% in the detection of SR. AFL and AF as shown in FIG. 5.

In addition, the presence of far field R wave interference also can result in misclassification of SR as AF. This problem may be addressed by, for instance, appropriate cross chamber blanking and careful positioning of the atrial lead to avoid far field R wave may minimize this problem.

5.1.7. Statistical Analysis

Continuous variables are expressed as mean ±1 standard deviation. The statistical comparisons were performed by Chi-square analysis and Student t test, as appropriate. To test the performance of the example embodiment of the disclosed discriminator, the sensitivity, specificity, and accuracy for detection of SR, AF, and AFL were calculated. (See Bland M. In: An Introduction to Medical Statistics, Chapter 15, Oxford University Press, 1996:273-276). Those with P values <0.05 were considered statistically significant.

5.1.7.1. Discriminator Performance

The performances of the new Bayesian Discriminator for the close-test and open-test data set are summarized in TABLE 2. A total of 3 episodes (4%) of false positive of AF detection occurred in 2 patients during SR due to the presence of far-field R wave sensing. All 50 episodes of sinus tachycardia were correctly identified as SR. The sensitivity, specificity and accuracy of rhythm detection for both close and open data set were similar. The overall sensitivity for detection of SR, AF and AFL is 97%, 97% and 94%, respectively; and the overall specificity for detection of SR, AF and AFL is 98%, 98% and 99%, respectively. The overall accuracy of detection of SR, AF and AFL is 98%, 97% and 98%, respectively (TABLE 2).

TABLE 2
Performances of the Bayserian Discriminator

| Rhythm Decision | | | | Performances | | |
|---|---|---|---|---|---|---|
| SR | AF | AFL | Total | Sensitivity | Specificity | Accuracy |

Close Data Set

Actual
rhythm

| | | | | | | | |
|---|---|---|---|---|---|---|---|
| SR | 70 | 2 | 0 | 72 | 97.2 | 98.6 | 98.2 |
| AF | 1 | 92 | 1 | 94 | 97.9 | 97.6 | 97.7 |
| AFL | 1 | 1 | 51 | 53 | 96.2 | 99.4 | 98.6 |

Open Data Set

Actual
rhythm

| | | | | | | | |
|---|---|---|---|---|---|---|---|
| SR | 47 | 1 | 0 | 48 | 97.9 | 97.9 | 97.9 |
| AF | 1 | 60 | 1 | 62 | 96.8 | 97.6 | 97.2 |
| AFL | 1 | 1 | 33 | 35 | 94.3 | 99.1 | 97.9 |

5.1.8. Main Findings

The results demonstrate that the features of intra-cardiac atrial electrograms, which included the regularity, rate, energy distribution, percent time of quiet interval and number of baseline reaching, are significantly different during SR, AFL, and AF. However, detection methods employing only one or few of these features have only limited sensitivity, specificity and accuracy for detection of SR, AFL, and AF. The disclosed Bayesian Discriminator based on the Bayes decision rule and five features of atrial electrograms, allows rapid on-line and accurate (98%) detection of SR, AFL, and AF with robust anti-noise performance. The disclosed discriminator requires a very short computing time. In an example embodiment, 250 ms are sufficient to make a decision for a rhythm episode of 1000 ms. As shown in the example section, the use of multiple features discrimination provides a much higher sensitivity, specificity and accuracy (all >94%) for rhythm detection than single or double features methods, as described above.

Clinically, as device therapies for atrial tachyarrhythmias become more sophisticated in their ability to deliver several modes of therapy, such as antitachycardiac pacing and defibrillation, depending on the specific rhythm, rapid and accurate detection of potentially tachycardias that can be terminated by pacing will be critical. Furthermore, accurate detection of SR from AFL and AF can also prevent inappropriate device therapy. The new Bayesian Discriminator described in this study, which is based on multiple features detection, can be easy implemented in the implantable device and provides rapid (>250 msec) and accurate (>97%) detection of AF, with robust anti-noise performance.

### 5.1.9. CONCLUSION

This disclosure encompasses new methods, systems, and devices for detecting arrhythmias and heart diseases based on multi-variate Bayes decision, which combine a plurality of different features of the intra-atrial electrogram. The described diagnostic tools enable superior overall sensitivity, specificity, and accuracy for rhythm detection than known single or double features methods as well as resistance to various ranges of noise.

However, citation of documents herein is not intended as an admission that any of the documents cited herein is pertinent prior art, or an admission that the cited documents are considered material to the patentability of the claims of the present application. Instead, they are intended to clearly describe the claimed invention. All statements as to the date or representations as to the contents of these documents are based on the information available to the applicant and does not constitute any admission as to the correctness of the dates or contents of these documents.

Although the present invention has been described in considerable detail with reference to certain preferred embodiments, other embodiments are possible. Therefore, the spirit and scope of the appended claims should not be limited to the description of the preferred embodiments contained herein. Modifications and variations of the invention described herein will be obvious to those skilled in the art from the foregoing detailed description and such modifications and variations are intended to come within the scope of the appended claims. Moreover, a number of references have been cited, the entire disclosures of which are incorporated herein by reference in their entirety.

What we claim:

1. A method for detecting a sinus rhythm of interest with aid of a computer executable instructions processing device based on electrophysiological information obtained from a subject via sensors coupled to the subject, comprising the steps of:
inputting a training data set as open-test data set of intra-atrial electrograms for evaluation, and
receiving a result in accordance with a set of estimated conditional probabilities of the sinus rhythm and a plurality of features.

2. A method according to claim 1 further comprising the steps of:
selecting the plurality of features of intra-atrial electrograms and a type of output,
inputting a new episode as close-test data set of intra-atrial electrograms for training, and
estimating the set of conditional probabilities for the plurality of features and the type of output in accordance with a multiple-index Bayesian discriminator algorithm from the close-test data set.

3. A method according to claim 2 further comprising the step of selecting additional features for estimating conditional probabilities.

4. A method according to claim 1 further comprising the step of modifying at least one estimated conditional probability from the set of estimated conditional probabilities with the open-test data set and the result.

5. A method according to claim 1 further comprising the step of diagnosing heart conditions in accordance with the result.

6. A method according to claim 2 further comprising the step of selecting a treatment to the subject corresponding to the result.

7. A method according to claim 2, wherein the plurality of features is selected from the group consisting of regularity, rate, energy distribution, percent time of quiet interval, and number of baseline reaching.

8. A method according to claim 2, wherein the type of output is selected from the group consisting of sinus rhythm, atrial flutter, and atrial fibrillation.

9. A method according to claim 2 further comprising the step of generating the result in less than five (5) seconds from receiving the open-test data set.

10. A device for providing an electrical signal in response to detecting a predetermined sinus rhythm with the aid of a set of computer executable instructions based on electrophysiological information obtained from sensors, for example, the device may comprise:
a set of power input terminals;
a data module for providing conditional probabilities of the predetermined sinus rhythm relative to a plurality of features;
an input for receiving electrophysiological information;
a member for providing the electrical signal for a modulating effect on heartbeats; and
at least one computer executable instructions processing unit.

11. The device of claim 10 wherein the predetermined sinus rhythm is selected from the group consisting of sinus rhythm, atrial flutter, and atrial fibrillation.

12. The device of claim 10 further comprising a module for identifying at least one feature from the group consisting of regularity, rate, energy distribution, percent time of quiet interval, and number of baseline reaching in the electrophysiological information.

13. The device of claim 10 further comprising a module or modules for updating a conditional probability of the predetermined sinus rhythm with the electrophysiological information.

14. The device of claim 10 wherein the electrical signal provided by the member belongs to the group consisting of the electrical signal or input to the chest wall that synchronizes the heart and allows the normal rhythm to restart, small electrical impulses to the heart muscle to maintain a suitable heart rate, electrical energy to the heart muscle to cause the heart to beat in a normal rhythm, and high radio-frequency energy through a special catheter to small areas of tissues that may be related to abnormal heart rhythms.

15. A computer readable media carrying thereon computer executable instructions for carrying out the steps of a method for detecting a sinus rhythm of interest based on electrophysiological information obtained from a subject via sensors coupled to the subject, the method comprising the steps of:
inputting an open-test data set of intra-atrial electrograms for evaluation, and
receiving a result in accordance with a set of estimated conditional probabilities of the sinus rhythm and a plurality of features.

16. The computer readable media of claim 15 further comprising computer executable instructions for carrying out the steps of:
selecting the plurality of features of intra-atrial electrograms and a type of output,
inputting a close-test data set of intra-atrial electrograms for training, and
estimating the set of conditional probabilities for the plurality of features and the type of output in accordance with a multiple-index Bayesian discriminator algorithm from the close-test data set.

17. The computer readable media of claim 16 further comprising computer executable instructions for carrying out the step of selecting additional features for estimating conditional probabilities.

18. The computer readable media of claim 15 further comprising computer executable instructions for carrying out the step of modifying at least one estimated conditional probability from the set of estimated conditional probabilities with the open-test data set and the result.

19. The computer readable media of claim 15 further comprising computer executable instructions for carrying out the step of diagnosing heart conditions in accordance with the result.

20. The computer readable media of claim 16 further comprising computer executable instructions for carrying out the step of providing a treatment to the subject corresponding to the result.

21. The computer readable media of claim 16 further comprising computer executable instructions wherein the plurality of features is selected from the group consisting of features such as regularity, rate, energy distribution, percent time of quiet interval, and number of baseline reaching.

22. The computer readable media of claim 16 further comprising computer executable instructions wherein the type of output is selected from the group consisting of sinus rhythm, atrial flutter, and atrial fibrillation.

23. The computer readable media of claim 16 further comprising computer executable instructions for carrying out the step of generating the result preferably in less than five (5) seconds from receiving the open-test data set.
